(19) United States
(12) Patent Application Publication
(10) Pub. No.: US 2013/0245491 A1
(43) Pub. Date: Sep. 19, 2013

(54) MULTI FUNCTIONAL MEDICAL DEVICE FOR SENSORY DIAGNOSTICS

(71) Applicant: Mauziar Nikzad, Bellevue, WA (US)

(72) Inventor: Mauziar Nikzad, Bellevue, WA (US)

(21) Appl. No.: 13/423,076

(22) Filed: Mar. 16, 2012

(51) Int. Cl. A61B 5/00

(52) U.S. Cl. 600/555

(57) ABSTRACT

A method and a device are disclosed for the diagnosis of sensory sensitivity in medical patients, including using a multifunctional medical device which may be utilized to test patient's skin sensitivity to a variety of stimuli and conditions such as heat or temperature, vibrations, electrical shock, pressure, pain, burning sensation, and the like. In some embodiments, the medical device includes a multifunction diagnostic head which can provide various stimuli at its tip, such as various temperatures, vibration, electrical shock, pressure, pain, and other stimuli which is detected by human skin sensory cells. In other embodiments, each of these stimuli may be provided by a separate removable diagnostic head. In still other embodiments, a removable diagnostic head may provide several, but not all, of the desired sensory stimuli listed above.

### TECHNICAL FIELD

This application relates generally to medical devices. More specifically, this application relates to a method and apparatus for assessing sensory perception.

### BRIEF DESCRIPTION OF THE DRAWINGS

The drawings, when considered in connection with the following description, are presented for the purpose of facilitating an understanding of the subject matter sought to be protected.

FIG. 1 shows an example otoscope with a removable diagnostic head;

FIG. 2 shows an example multifunctional medical diagnostic device with a removable diagnostic head;

FIG. 3 shows an example electronic thermo-cooling device;

FIG. 4A shows an example removable diagnostic head configured to test pressure sensation; and

FIG. 4B shows an example removable diagnostic head configured to test pain sensation.

### DETAILED DESCRIPTION

While the present disclosure is described with reference to several illustrative embodiments described herein, it should be clear that the present disclosure should not be limited to such embodiments. Therefore, the description of the embodiments provided herein is illustrative of the present disclosure and should not limit the scope of the disclosure as claimed. In addition, while the following description references a thermal sensitivity diagnostic head, it will be appreciated that the disclosure may be used with other types of sensory diagnostic heads such as pressure sensitivity, burning sensitivity, and the like.

Briefly described, a method and a device are disclosed for the diagnosis of sensory sensitivity in medical patients, including using a multifunctional medical device which may be utilized to test patient's skin sensitivity to a variety of stimuli and conditions such as heat or temperature, vibrations, electrical shock, pressure, pain, burning sensation, and the like. In some embodiments, the medical device includes a multifunction diagnostic head which can provide various stimuli at its tip, such as various temperatures, vibration, electrical shock, pressure, pain, and other stimuli which is detected by human skin sensory cells. In other embodiments, each of these stimuli may be provided by a separate removable diagnostic head. In still other embodiments, a removable diagnostic head may provide several, but not all, of the desired sensory stimuli listed above.

Many medical conditions or diseases have symptoms which are manifested on skin, particularly on finger tips or toe tips. Hence, such conditions or diseases may be diagnosed, at least partially, by assessing sensations a patient feels on his finger tips or toe tips as a result of measured or controlled stimuli applied to the finger tip. The utility of finger tip sensation tests are not limited to diagnosing diseases, but may be useful in a general health assessment, for example, during a physical exam.

Some of the conditions or diseases which may have sensory symptoms include diabetes, hypothyroidism, blood circulation problems, nervous system problems such as neuropathy (dying nerves), and the like.

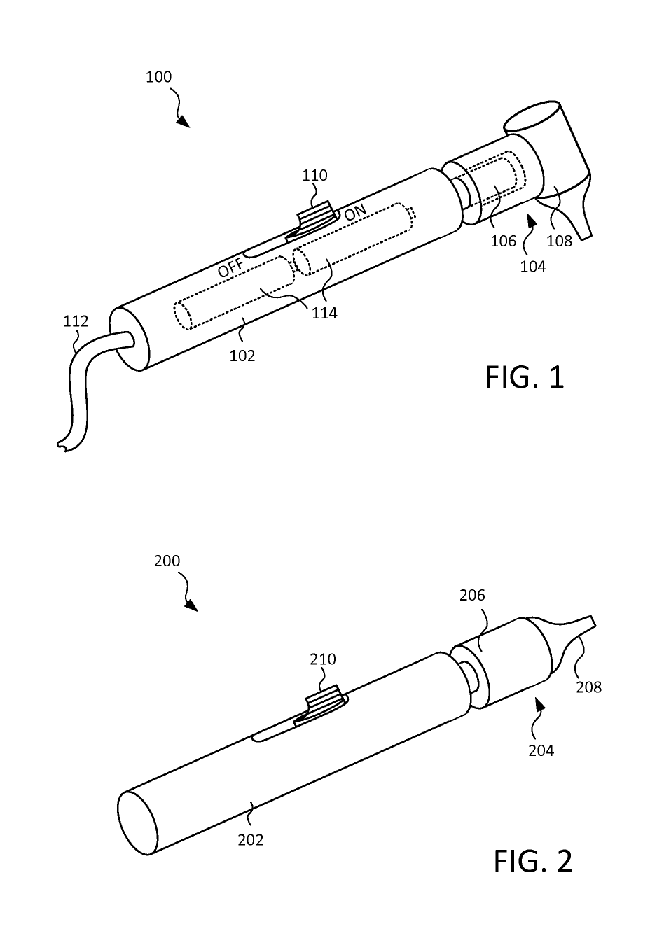

FIG. 1 shows an example otoscope with a removable diagnostic head. In some embodiments, otoscope 100 includes handle 102, ON/OFF switch 110, removable diagnostic head 104 including an attachment base 106, and diagnostic tip 108. Otoscope 100 may be powered, if needed, by batteries 114 or power cord 112.

Otoscope is typically used for diagnosing health problems of ear, nose, and throat by looking through a narrow, lighted, and sometimes magnified tip (using a lens) at the inside of ear, nose, or throat canals. In some embodiments, diagnostic head 104 may be removable to allow the attachment of the appropriate diagnostic tips with different shapes and functionalities. For example, a diagnostic tip for looking inside the ear may be different from one for looking inside the nose or throat.

Because Otoscope is a common hand-held medical device used in almost all physician's offices, it may be a good platform to use for other common diagnostic tests, such as testing various sensations on finger tips or toe tips. An otoscope provides a handle, a power source, and an interface for attaching different heads, which are all useful features for a general hand-held diagnostic device.

FIG. 2 shows an example multi functional medical diagnostic device with a removable diagnostic head. In some embodiments, medical diagnostic device 200 includes handle 202, removable diagnostic head 204 including attachment base 206 and diagnostic tip 208. Attachment base 206 is generally used to couple the diagnostic head to handle 202 of medical diagnostic device 200 using various coupling techniques, such as screw and threads, twist-lock, floating ball lock, and the like. In other embodiments, the medical diagnostic device may be a single integrated piece without a removable or separate diagnostic head. Those skilled in the art will appreciate that many other configurations of such medical device may be made without departing from the spirit of the present disclosures. For example, the handle may be similar to a gun-handle, rather than a straight one.

In various embodiments, diagnostic tip 208 may be configured to perform one or more testing functions including thermal (hot and cold), pressure, pain, electrical shock, burning, and other similar sensations.

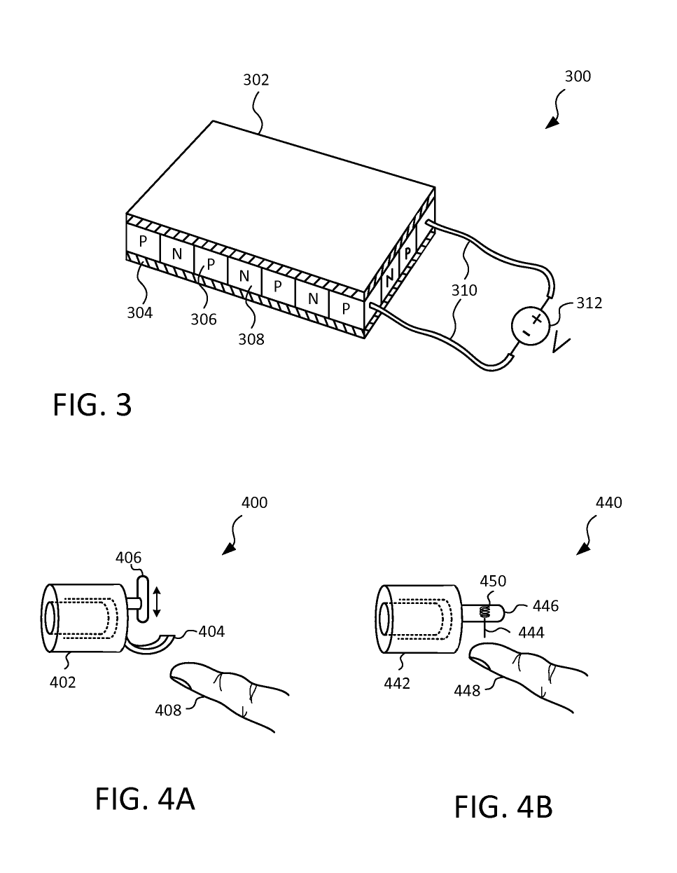

In various embodiments, diagnostic tip 208 is configured to provide thermal stimulus. In some embodiments, a preset temperature is provided by the tip upon turning on of the device. For example, different tips may be preset to different temperatures, hotter or colder than human body's temperature. In other embodiments, a temperature control slider or knob is provided to set the tip temperature to a desired value, using a built-in temperature control unit to regulate the temperature. The heat or cold may be provided using an thermoelectric heater and/or cooler, further described below with respect to FIG. 3. In some embodiments two or more thermoelectric coolers may be used for quick switching between hot and cold or for providing more heat capacity to heat or cool an area.

In application, diagnostic tip 208 is touched to the fingertip or other skin area of the patient to test the patient's sensory sensitivity to the particular stimulus, such as heat or cold. In various embodiments, the value or level of the stimulus may be recorded to be included in patient's records or consistently characterize the patient's sensitivity to the particular stimulus. For example, a patient may be sensitive to heat sensation only after the thermal stimulus is set to a value above 100° F., or cold sensation below 48° F. Similarly, other stimuli, such as vibration, pressure or electrical shock, may be set to particular values.

In some embodiments, diagnostic tip 208 may be coupled with a vibration generating component, such as a small electric motor, or armature. The shaft of the armature may be coupled to a cam in an off-center position to convert the rotational motion of the shaft to a vibratory motion. The vibration thus generated at the diagnostic tip may be used as a stimulus to test touch sensitivity. In other embodiments, a rotating brush or other similar articles may be used to stimulate the sense of touch.

In some embodiments, diagnostic tip 208 may be coupled to an electrical voltage and/or current source, such as a chargeable capacitor housed within the diagnostic head 204 or handle 202, to furnish, upon the activation of the diagnostic tip, a measured electrical charge to the patient's finger to test the patient's sensitivity to such stimuli. In some embodiments, the capacitor may be charged to a fixed and predetermined level, while in other embodiments, the charge of the capacitor may be programmable and/or adjustable.

In various embodiments, the type and value of the sensory stimulus applied by the medical diagnostic device is recorded in a memory module embedded in the medical diagnostic device. In other embodiments, the type and value of the sensory stimulus applied by the medical diagnostic device is wirelessly transmitted to a recording device external to the medical diagnostic device, such as a computer.

FIG. 3 shows an example electronic thermo-cooling device. In various embodiments, electronic thermo-cooling device 300 includes a hot-side thermal surface 302 and a cold-side thermal surface 304 enclosing between them P-N junctions having positively doped, or P type, and negatively doped, or N type, semiconductors. Conductors 310 connected to Direct Current (DC) voltage source 312 cause a temperature differential to be established between the two thermal surfaces, making one surface hotter than the other and both surfaces hotter or colder than the ambient temperature. Such an arrangement essentially establishes a heat pump which pumps thermal energy from one conducting surface to the other, making the heat source surface colder and the heat target surface hotter.

In various embodiments, the thermo-cooling device is small enough that it may be built-in at or very near the diagnostic tip. This way, thermal efficiency is preserved and the desired temperature may be achieved more quickly without loss of heat.

FIG. 4A shows an example removable diagnostic head configured to test pressure sensation. In various embodiments, removable diagnostic head 400 includes an attachment base 402, finger cradle 404, and pressure applicator component 406 used to apply a pressure to finger 408.

In various embodiments, pressure applicator component 406 is configured to move in a direction to exert a pressure on finger 408. For example, the pressure applicator may be a rod which can move up and down over the finger cradle to apply a measured pressure onto finger 408. A physician may then ascertain when the finger senses the pressure and whether its sensitivity is within normal medical range. In various embodiments, pressure head 400 may be preset to a particular fixed pressure so that once the pressure applicator is released or activated, it exerts a particular amount of force or pressure onto the finger. In other embodiments, pressure head 400 may include an adjustment control to variably set the exerted pressure to a desired level. Such adjustment may implemented using a spring tensioner arrangement, a wedge arrangement or other known mechanical techniques. The pressure adjustment mechanism may be calibrated to know what pressure it corresponds to.

Cradle 404 is used to support the finger and also to distribute the pressure evenly on the back of the finger (where fingernail is located) so the sensory focus of the patient is on the front of the finger facing pressure applicator 406.

FIG. 4B shows an example removable diagnostic head configured to test pain sensation. In various embodiments, diagnostic head 440 includes attachment base 442, diagnostic tip 446, pin 444 for lightly puncturing or pricking finger 448, and spring 450.

In various embodiments, pin 444 is coupled with spring 450 which, upon activation of diagnostic tip 446, lounges pin 444 towards finger 448 with a particular force and to a particular extent or distance to prick the finger in order to test pain sensitivity. In other embodiments, pin 444 may be driven using other techniques such as magnetic force, pneumatic force (like an air cylinder and piston arrangement), or other suitable techniques for driving the pin. In some embodiments, the force and extension distance of the pin towards the finger are predetermined, while in other embodiments, they are adjustable.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While the present disclosure has been described in connection with what is considered the most practical and preferred embodiment, it is understood that this disclosure is not limited to the disclosed embodiments, but is intended to cover various arrangements included within the spirit and scope of the broadest interpretation so as to encompass all such modifications and equivalent arrangements.

What is claimed is:

1. A medical diagnostic device comprising:
a handle; and
a diagnostic head including an attachment base and a diagnostic tip, wherein the diagnostic tip is configured to provide a sensory stimulus.

2. The medical diagnostic device of claim 1, wherein the handle is powered.

3. The medical diagnostic device of claim 1, wherein the handle is powered by one of a battery and a power cord.

4. The medical diagnostic device of claim 1, wherein the diagnostic head is removable.

5. The medical diagnostic device of claim 1, wherein the diagnostic tip is configured to provide one of a heat, cold, vibration, pressure, electrical shock, and pain stimuli.

6. The medical diagnostic device of claim 1, wherein the diagnostic tip is configured to provide a heat or cold stimulus using an electronic thermo-cooler device operating as a heat pump.

7. The medical diagnostic device of claim 1, wherein the diagnostic tip is configured to provide pressure stimulus using a pressure applicator.

8. The medical diagnostic device of claim 1, wherein the sensory stimulus has a measured value which is adjustable.

9. A medical diagnostic device comprising:
a powered handle; and
a diagnostic head including an attachment base and a diagnostic tip, wherein the diagnostic tip is configured to provide a sensory thermal stimulus.

10. The medical diagnostic device of claim 9, wherein the powered handle is powered by battery or by an electrical cord.

11. The medical diagnostic device of claim 9, wherein diagnostic head is removable.

12. The medical diagnostic device of claim 9, wherein the diagnostic tip is further configured to provide other sensory stimulus in addition to the sensory thermal stimulus.

13. The medical diagnostic device of claim 9, wherein the value of the sensory thermal stimulus is recorded.

14. The medical diagnostic device of claim 9, wherein the sensory thermal stimulus is provided using an electronic thermo-cooler device.

15. The medical diagnostic device of claim 9, wherein the sensory thermal stimulus has an adjustable value.

16. A medical diagnostic device comprising:
a powered handle; and
a removable diagnostic head including an attachment base and a diagnostic tip, wherein the diagnostic tip is configured to provide a plurality of sensory stimuli.

17. The medical diagnostic device of claim 16, wherein the plurality of sensory stimuli include two or more of heat, cold, vibration, pressure, electrical shock, and pain stimuli.

18. The medical diagnostic device of claim 16, wherein at least one of the plurality of sensory stimuli has an adjustable value.

19. The medical diagnostic device of claim 16, wherein types and values of at least some of the plurality of sensory stimuli are recorded.

20. The medical diagnostic device of claim 16, wherein one of the plurality of sensor stimuli is a sensory thermal stimulus provided using an electronic thermo-cooler device.
